(19) United States
(12) Patent Application Publication
(10) Pub. No.: US 2004/0092879 A1
(43) Pub. Date: May 13, 2004

(54) Safety introducer apparatus and method therefor

(75) Inventors: Mark C. Kraus, Independence, MN (US); Valerie Glazier, Minnetonka, MN (US); Todd Latterell, Crystal, MN (US)

Correspondence Address: Schwegman, Lundberg, Woessner & Kluth, P.A., P.O. Box 2938, Minneapolis, MN, 55402, US

(73) Assignee: MedAmicus, Inc.

(21) Appl. No.: 10/692,614

(22) Filed: Oct. 24, 2003

Related U.S. Application Data
(63) Continuation-in-part of application No. 09/707,162, filed on Nov. 6, 2000, now Pat. No. 6,641,564.
(63) Continuation-in-part of application No. 10/403,265, filed on Mar. 26, 2003.

(51) Int. Cl. A61M 5/178

(52) U.S. Cl. 604/158; 606/108

(57) ABSTRACT

An introducing apparatus is provided which includes an elongate tubular sheath extending from a sheath proximal end to a sheath distal end. The sheath has a bore sized to receive a dilator therethrough. The sheath further includes at least one tab extending away from a longitudinal axis of the sheath. The dilator extends from a dilator proximal end to a dilator distal end. The introducing apparatus further includes a needle disposed within the dilator, and retractably coupled with the dilator. One or more portions of the needle is flexible.

### RELATED APPLICATIONS

This application is a continuation-in-part of the following: U.S. patent application Ser. No. 09/707,162, filed on Nov. 6, 2000, entitled “SAFETY INTRODUCER APPARATUS AND METHOD THEREFOR”, and U.S. patent application Ser. No. 10/403,265, filed on Mar. 26, 2003, entitled “SAFETY INTRODUCER ASSEMBLY AND METHOD”, the specifications of which are incorporated herein by reference.

### TECHNICAL FIELD

The present application generally relates to introducers and introducing assemblies. Specifically, it relates to a safety introducer.

### BACKGROUND

Introducer devices provide for access to the venous system and are employed for inserting medical devices such as catheters, guidewires, leads, infusion ports, dialysis ports, dialysis catheters, and others. A typical procedure for gaining access to the central venous system or the arterial system with an introducer is the Seldinger Introduction Method. The Seldinger Method provides for insertion of a needle into the vasculature of a patient. Once the needle is in the vessel, the physician aspirates the needle to assure that the needle is in the vessel, and to draw out air present in the bore of the needle. The syringe is removed and discarded. A guide wire is inserted through the needle, and the needle is removed over the guide wire. The introducer, which includes a dilator and the sheath, is placed over the guidewire and inserted into the vessel. With the introducer and wire guide in the vessel, the dilator and wire guide are removed leaving only the sheath in the vessel. The desired medical device is implanted through the bore of the sheath. The sheath is optionally removed from the medical device.

Any time a needle is used it can cause transmission of various pathogens, most notably the Human Immune Virus (HIV), due to an accidental needle stick of an uninfected person after the needle is withdrawn from the patient, or due to re-use of a needle. Furthermore, the Seldinger Method requires numerous steps, resulting in extra costs, potential trauma, and/or pain for a patient.

Accordingly, what is needed is an introducer and dilator which can eliminate needle re-use or inadvertent needle sticks. What is also needed is an introducer assembly which does not distract or interfere with the implantation process.

### SUMMARY

An introducing apparatus is recited herein and includes a tubular sheath and a dilator extending therethrough. A needle is disposed within the dilator, where at least a portion of the needle is flexible, and the needle is retractably disposed within the dilator. Optionally, the needle distal end, and/or the needle intermediate portion is more flexible than the dilator. In yet another option, the introducing apparatus includes features that prevent re-extension of the needle distal end.

The introducing apparatus beneficially provides a safety introducer, which allows for the needle to be safely retracted within the dilator after its use, and optionally prevents re-use of the same needle, for example on another patient.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 illustrates a perspective view of an introducing apparatus as constructed in accordance with one embodiment;

FIG. 2 illustrates a perspective view of a disassembled introducing apparatus as constructed in accordance with one embodiment;

FIG. 3A illustrates side cross-sectional view of a portion of an introducing apparatus as constructed in accordance with one embodiment;

FIG. 3B illustrates side cross-sectional view of a portion of an introducing apparatus as constructed in accordance with one embodiment;

FIG. 4 illustrates a perspective view of a disassembled introducing apparatus as constructed in accordance with another embodiment;

FIG. 5 illustrates a perspective view of a portion of an introducing apparatus as constructed in accordance with one embodiment;

FIG. 6 illustrates a perspective view of a portion of an introducing apparatus as constructed in accordance with one embodiment;

FIG. 7 illustrates a side elevational view of an introducing apparatus as constructed in accordance with one embodiment.

FIG. 8 illustrates a cross-sectional view of a dilator and needle assembly as constructed in accordance with one embodiment.

FIG. 9 illustrates a valve and stop cock assembly for use in arterial applications.

FIG. 10 illustrates a cross-sectional view taken along A-A of FIG. 11, of the introducer apparatus as constructed in accordance with one embodiment.

FIG. 11 illustrates a side elevational view of an introducer apparatus as constructed in accordance with one embodiment.

FIG. 12 illustrates a cross-sectional view of an actuator assembly constructed in accordance with one embodiment.

FIG. 13 illustrates a side elevational view of a needle as constructed in accordance with another embodiment.

FIG. 14 is a cross-sectional view illustrating an introducer assembly constructed in accordance with one embodiment.

FIG. 15 is a cross-sectional view illustrating an introducer assembly constructed in accordance with one embodiment.

FIG. 16 is a cross-sectional view illustrating an dilator assembly constructed in accordance with one embodiment.

FIG. 17 is a perspective view illustrating an introducer assembly constructed in accordance with one embodiment.

FIG. 18 is a cross-sectional view illustrating an introducer assembly constructed in accordance with one embodiment.

FIG. 19 is a perspective view illustrating an introducer assembly constructed in accordance with one embodiment.

FIG. 20 is a cross-sectional view illustrating an introducer assembly constructed in accordance with one embodiment.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

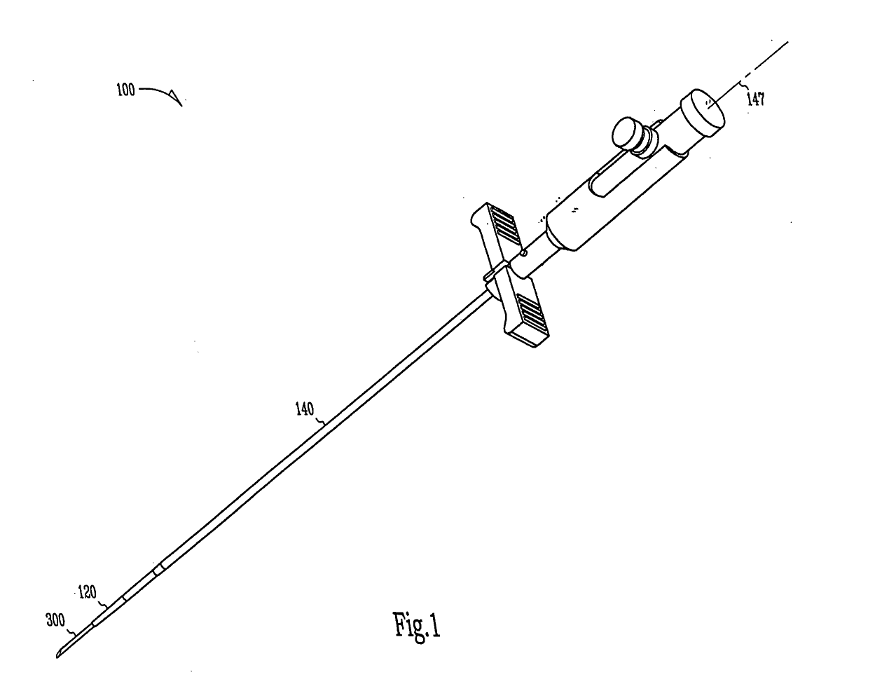

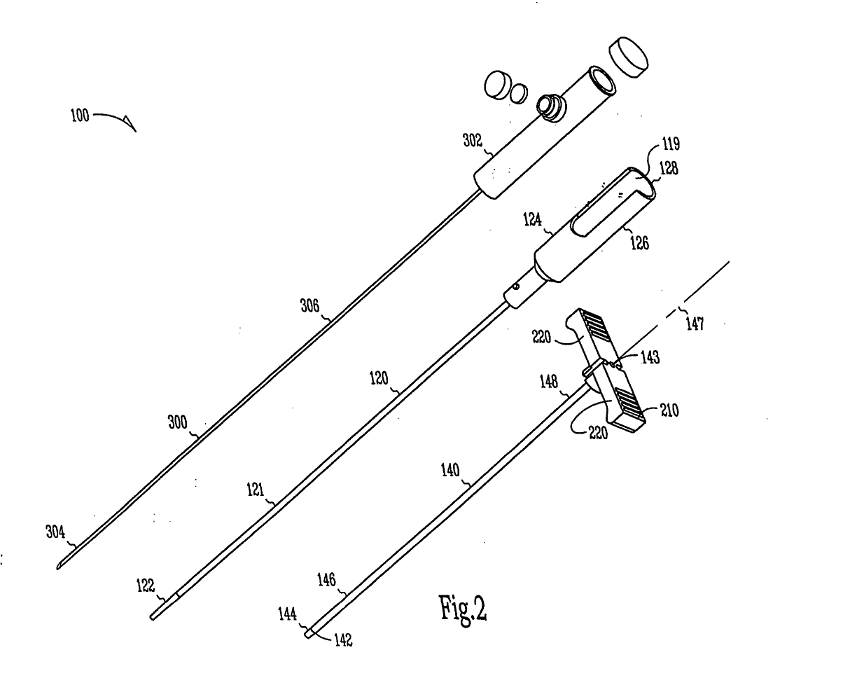

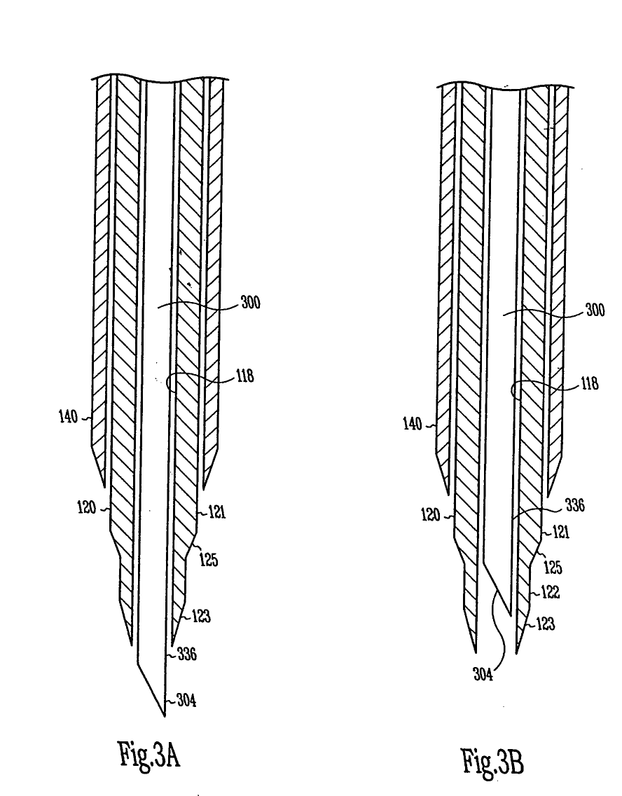

An introducer assembly 100, as shown in FIGS. 1 and 2, includes generally a sheath 140 and a dilator 120 through the sheath 140, and a needle 300 disposed within the dilator 120. The dilator 120 and the needle 300 allow for the introducer assembly 100 to be introduced into a vessel of a patient. The dilator 120 extends from a dilator distal end 122 to a dilator proximal end 124, where the dilator distal end 122 is insertable into a patient. Disposed between the dilator distal end 122 and the dilator proximal end 124 is a dilator intermediate portion 121. The dilator distal end 122 optionally ends in a tapered end 123, as shown in more detail in FIGS. 3A and 3B. In another option, the dilator distal end 122 has a tapered end 123, and a second tapered portion 125, where the second tapered portion 125 is disposed in the dilator intermediate portion 121. In one option, an outer surface 336 of the needle 300 directly abuts an inner surface 118 of the dilator, thereby allowing the introducing assembly 100 to have a thin outer diameter.

Referring again to FIGS. 1 and 2, at the dilator proximal end 124 is a hub 126 having a bore 128 therethrough. The dilator 120 also includes a passage 119 therethrough, aligned with the bore 128, which allows the dilator 120 to be inserted over the sheath 140. In a further option, the dilator 120 includes a blood flashback chamber 180, which is coupled with the hub 126 of the dilator 120, as shown in more detail in FIG. 8. The blood flashback chamber 180 is filled with blood as the physician inserts the needle 300 of the introducer assembly 100 within a vessel of a patient. One end of the flashback chamber 180 is sealed by a gas permeable filter 182 which allows air to pass therethrough, although prevents blood to pass from the flashback chamber 180.

During use of the assembly 100 (FIG. 1), once the needle has entered a blood pressure environment, the pressure will cause the blood to exit the hole in the blood vessel made by the needle 300. The blood enters a distal end 304 of the needle 300, and travels through the passage 143 of the sheath 140, which has a lower pressure than blood pressure. The blood will travel from the distal end 304 of the needle 300 to the proximal end 302 of the needle 300 and into the flashback chamber 180 located at the proximal end 302 of the needle 300. The blood pressure, which is greater than the ambient pressure outside of the blood vessel, will force the air in the needle 300 out of the gas permeable filter 182 coupled with the flashback chamber 180.

The gas permeable filter 180 is in contact with the ambient environment outside of the needle 300, to which the air escapes. Once all of the air has been pushed out of the needle 300 by the blood pressure, the blood appears in the flashback chamber 180. The filter 180 prevents blood from exiting the chamber 180. The flashback chamber 180 is visible to the user, indicating to the user that the needle 300 has been aspirated, and that access to the blood vessel has been obtained. In another option, the flashback chamber 180 further includes a luer fitting 305. The user optionally attaches a syringe to the luer fitting 305, and aspirates the needle 300 using the syringe.

The dilator 120 is sized to be received by the sheath 140 therein. The sheath 140 allows for additional instruments to be inserted therethrough and inserted into the patient. The sheath 140 includes various types of sheaths, for instance, the sheath 140 can comprise a sheath which has a strengthening braid of material. Alternatively, the sheath 140 includes those which are modified to prevent bends in the elongate sheath. The sheath 140 is defined in part by a longitudinal axis 147, and the sheath 140 extends from a sheath distal end 142 to a sheath proximal end 148. The sheath 140 is coaxial with the dilator 120, and optionally the needle 300, where they each share the same longitudinal axis 147. The distal end 142 of the sheath 140 is first inserted into the patient and the proximal end 148 remains outside of the patient. Near the distal end 142 is an optional tapered portion 144 which provides a transition to a cylindrical portion 146. The sheath 140 also includes a passage 143 therethrough, where the passage 143 is substantially aligned with the longitudinal axis 147 of the sheath 140. The passage 143 allows for the introduction of the dilator 120 therethrough. After the introducer assembly 100 has been inserted into a patient, and the dilator 120 is removed, other medical instruments can be easily inserted into and through the sheath 140, and introduced into the patient.

The sheath 140 includes at least one tab 210 which extends radially outward from the sheath 140. In one embodiment, the sheath 140 includes two tabs 220 which are disposed 180 degrees from each other. Optionally, tab break lines 222 (FIG. 5) are disposed between along the sheath 140, for instance between the two tabs 220 are tab break lines 222 (FIG. 5).

In another option, the sheath 140 is splittable such that the sheath 140 is separable into two or more components. The sheath 140 is separable or splittable away from instruments inserted therethrough which prevents disruption to or removal of instruments or devices which have been inserted through the sheath 140. The splittable sheath 140 is separable from the instruments inserted therethrough, where no damage occurs to the instruments during the removal of the sheath 140. For example, in one option, the sheath 140 includes at least one score line 141, as shown in FIG. 5. The sheath 140 is externally scored, and optionally two scores 141 are 180 degrees from each other. The scores 141 are aligned with the optional tab break lines 222 such that the tab break lines 222 and the scores 141 are disposed between the two tabs 220. Alternatively, the sheath 140 is splittable using a slitting device, a rip cord or strengthening strip running along the longitudinal length of the sheath, a weakening which allows the introducer to be ripped apart, or other techniques which allow the sheath 140 to separate without damage to an instrument inserted therethrough, or without disruption to the procedure.

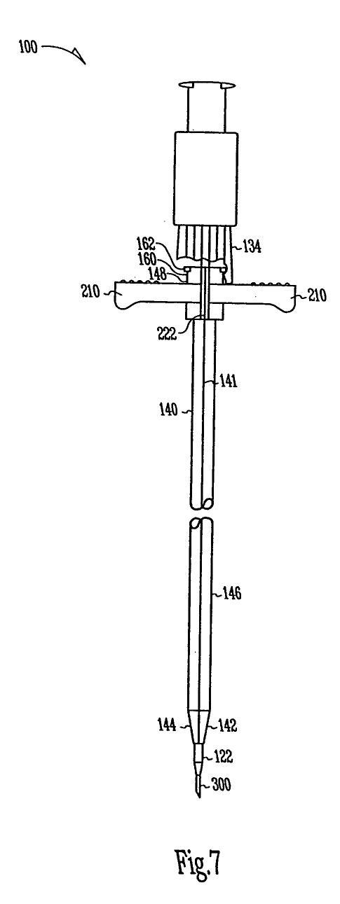

It should be noted that the introducer assembly 100 can be used for both venous and arterial applications. For arterial applications, it may not be necessary to remove the sheath while a medical instrument is inserted therethrough. In one option, the sheath 140 is not separable. FIG. 9 illustrates an example of a valve 139 to be used with the introducer assembly 100, for example, for arterial applications. The introducer 100 is disposed through the valve 139, and the valve 139 is coupled with a proximal end 148 of the sheath 140. In a further option, a stop cock 137 is coupled with the valve 139. The stop cock 137 allows for the introduction of fluids therethrough and into the patient.

Referring again to FIGS. 1 and 2, as mentioned above, a needle 300 is disposed within the dilator 120. In one option, the needle is retractably coupled with the dilator. The needle 300 extends from a needle proximal end 302 to a needle distal end 304, and includes a needle intermediate portion 306 therebetween. The needle 300 is coaxial with the sheath 140 and the dilator 120 (FIG. 1). For instance, a longitudinal axis of the needle 300 is aligned with the longitudinal axis 147 of the sheath 140 (FIG. 1), when the needle 300 is in the extended and retracted positions.

In one option, the needle distal end 304 is echogenic, which allows for the physician to view the needle 300 during the process of implanting the medical device. The needle 300 is movably disposed within the dilator 120, as shown in FIGS. 3A and 3B, and as further discussed below. The distal end 304 of the needle extends out from the dilator distal end 122 in a first position (FIG. 3A). The needle distal end 304 is retracted within the dilator 120 in a second position (FIG. 3B), and the needle 300 is retractably coupled with the dilator.

The needle 300, in one option, is flexible along a portion of or the entire needle, allowing the needle to be inserted further into a vessel than conventional needles. For example, the needle 300 is formed of flexible material, such as nitinol. In one option, the needle 300 is formed of a unitary structure of nitinol. In another option, at least a portion of the needle 300 is flexible. For instance, a portion of the needle 300 is formed of a flexible material such as nitinol. In another option, at least a portion of the needle 300 is flexible as it includes a first portion 305 formed of a spring coil 307, as shown in FIG. 13. In yet another option, the spring coil 307 is coated with a material, such as Teflon. Other coatings which maintain flexibility of the needle 300 are suitable as well. In yet a further option, a second portion 308 of rigid or semi-rigid material is coupled with the spring coil 307. The second portion 308, in one option, has a length 309 of about 0.5 inches.

Since the needle is flexible, the guidewire is no longer necessary to introduce devices into a patient. This allows for the assembly to be manufactured more cost effectively, and further allows for a faster introduction process. In another option, only the needle distal end 304 and/or the needle intermediate portion 306 is flexible. Optionally, the needle 300 has the same or more flexibility than the dilator 120. The needle 300 is flexible enough to permit insertion of the needle 300 through the right side subclavian vein into the superior venacava without kinking or causing the dilator to perforate the vein. In another option, the needle 300 is flexible enough such that it is insertable around the aortic bifurcation without kinking or causing the dilator to perforate a femoral artery. In a further option, the needle 300 is flexible enough such that it can be bent into a circle having a 0.5 inch radius. In addition, the needle 300 has sufficient flexibility and column strength to be pushed through the vasculature by a user without kinking the needle 300.

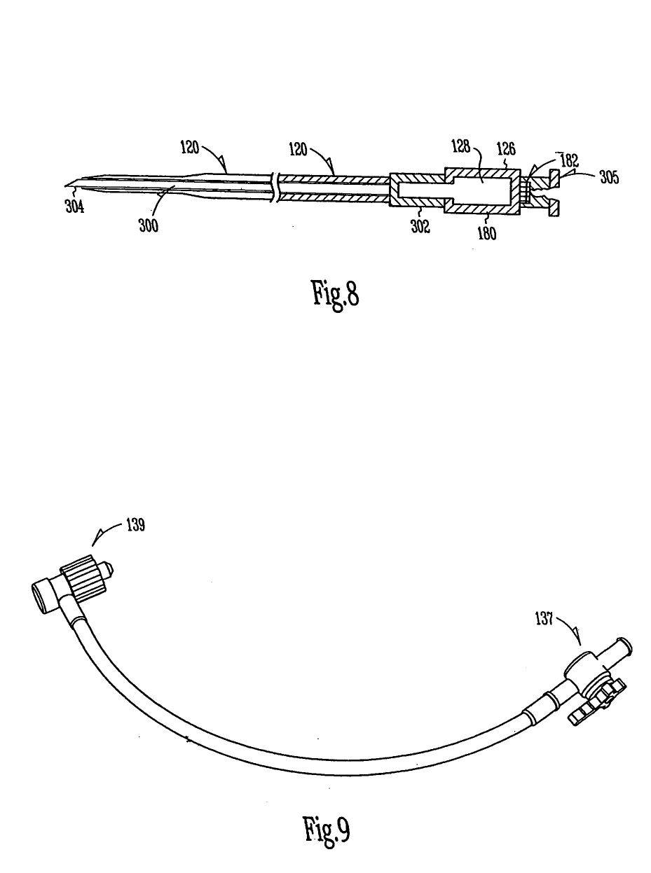

FIGS. 10 and 11 illustrate the needle 300, the sheath 140 and the dilator 120 in greater detail. The needle 300 is attached to a needle hub 340, which is retractably coupled with the dilator 120. A rear barrel 344 is coupled with the dilator 120, where the rear barrel 344 does not move relative to the dilator 120. A bias member 342, such as a spring, is disposed within the hub 126 of the dilator 120, and biases the needle hub 340 and the needle 300 toward the proximal end of the assembly 100 toward a retracted position. A needle retainer 346 releasably retains the needle hub 340 against the bias of the bias member 342.

The rear barrel 344 has a hollow central bore, and includes at least one locking aperture 348 in a sidewall 350 of the rear barrel 344. The proximal end 352 of the rear barrel 344 is generally open for receiving the needle hub 340 and a connector hub 354 therein, where the connector hub 354 in one option comprises a luer fitting. The rear barrel 344 further includes a stop 356 which limits displacement of the needle 300, and limits the retraction of the needle 300.

The needle hub 340 is generally cylindrical and is coupled with the needle 300. The needle retainer 346 includes an actuator 358. In one option, the actuator 358 comprises a deformable arm. Coupled with at least a portion of an actuator 358 is an actuator button 360. The actuator button 360 is received within the locking aperture 348 when the needle 300 is disposed in the retracted position. The actuator button 360 is configured to cooperate with the locking aperture 348 in the rear barrel 344, to releasably engage the needle hub 340 with the rear barrel 344.

The needle 300 is operable between a projecting position illustrated in FIG. 3A and retracted position illustrated in FIG. 3B, and as further discussed below. In one example, the actuator button 360 allows a user to move the needle 300 from an extended position (FIG. 10) to a retracted position (FIG. 3B). A flat 362 of he actuator button 362 is engaged with a portion of the rear barrel 344 and retains the needle 300 in an extended position (FIG. 10). Once the actuator button 360 is depressed toward a longitudinal axis of the assembly 100, the flat 362 is released from the rear barrel 344, and the bias member 342 forces the needle 300 into a retracted position (FIG. 3B). There are other ways of retracting the needle 300, for example, as further discussed below.

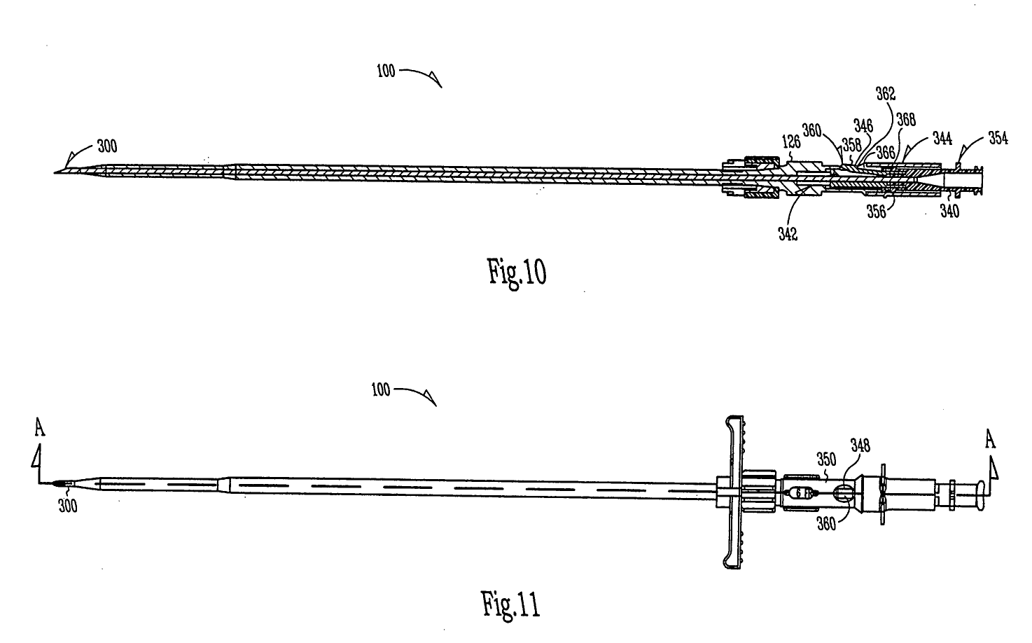

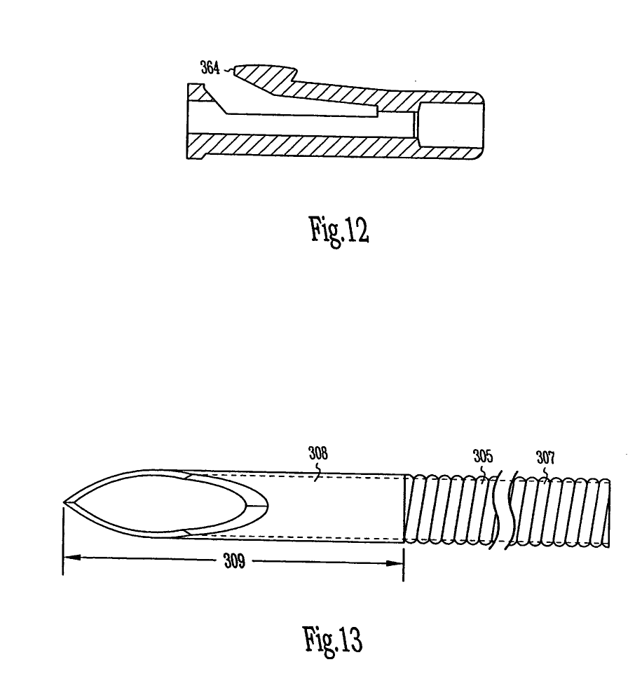

The assembly 300 optionally further provides for preventing re-extension of the needle 300 after retraction of the needle 300 within the dilator 120, so that a contaminated distal end 304 of the needle 300 is not exposed and cannot be re-exposed. In one option, actuator 358 assists in preventing the re-extension of the needle 300, where the actuator 358 is shown in FIG. 12 in greater detail. The actuator button 360 includes a shoulder 364 that engages a flange 366 on an interior surface of the rear barrel 344, as shown in FIG. 10. As the needle 300 is retracted within the dilator 120, the needle retainer 346 moves past the flange 366, and flexes radially outwardly when it is displaced past the flange 366 and into the larger inner diameter 368. The shoulder 364 of the actuator button 360 abuts up against the flange 366 and prevents re-extension of the needle 300, if a user attempts to re-extend the needle 300.

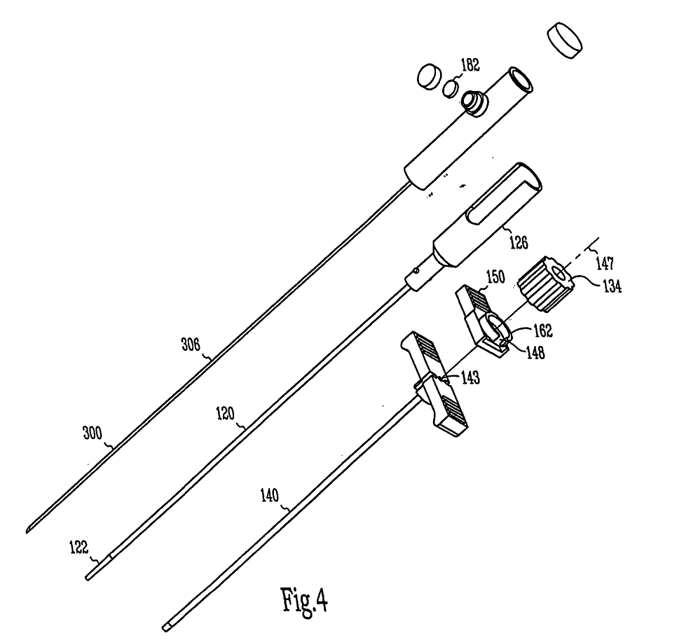

In a further option, the sheath 140 includes a valve assembly 150 coupled therewith, as shown in more detail in FIGS. 4 and 5. Optionally, the valve assembly 150 is movably coupled with the at least one tab 210, where the valve assembly 150 is movable relative to a top surface 212 of the at least one tab 210. In another example, the valve assembly 150 is slidingly coupled with the at least one tab 210.

The valve assembly 150 includes a seal 152 and a valve support member 154. The valve support member 154, in combination with the seal 152, provide a hemostatic valve which seals against instruments which are disposed therethrough. In addition, the valve assembly 150 provides a seal for the passage 142 of the sheath 140, where little or no air is allowed to enter the vessel of a patient. The seal 152, in one option, comprises a membrane. A further option is that the seal 152 includes a slitted portion 156 therein. The slitted portion 156 can include, but is not limited to, a number of different options such as a slit, a partial slit, a line of weakness, or a perforated line. In yet another option, the seal 152 comprises multiple sealing components, for instance, which are disposed adjacent to one another.

The valve support member 154 retains the seal 152. In addition, the valve support member 154 is coupled with the sheath 140, and allows for the valve assembly 150 to move relative to the sheath 140. The valve assembly 150 moves relative to the sheath in many different manners.

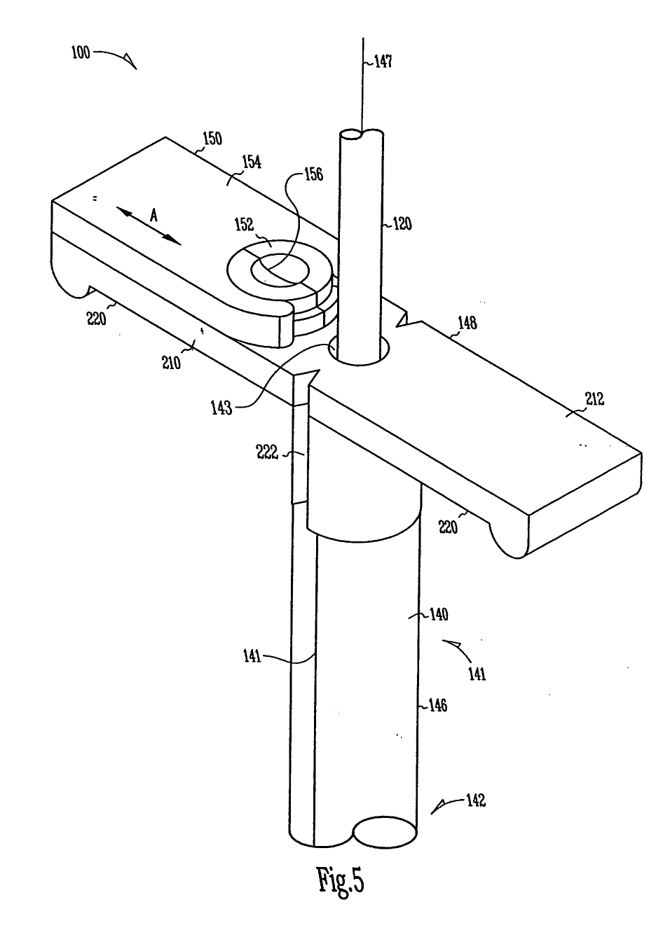

In one example, the valve support member 154 is adapted to slide along a longitudinal axis of the at least one tab. The valve support member 154, in one option, is disposed around only a portion of the seal 152. In another option, the valve support member 154 flexes as an instrument is disposed through the seal 152. The movable valve assembly 150 is adapted to slide from a first position, as shown in FIG. 5, to a second position, as shown in FIG. 6. In the first position, the movable valve assembly 150 is disposed through the longitudinal axis of the sheath, sealing the passage of the sheath 140. In the second position, the movable valve assembly 150 is disposed away from the longitudinal axis of the sheath. As shown in the drawings, the movably valve assembly 150 can be moved from the first position to the second position, and from the second position to the first position while an instrument is disposed within the sheath 140, allowing for increased flexibility.

In another example, the movable valve assembly 150 is adapted to rotate about a hinge point on the at least one tab of the sheath. As the movable valve assembly 150 rotates, the valve assembly 150 slides on a top surface of the at least one tab. In another embodiment, the movable valve assembly 150 is adapted to rotate about a hinge point on the at least one tab. As the movable valve assembly 150 rotates about the hinge point, at least a portion of the valve assembly 150 is lifted away from the top surface of the at least one tab. The movable valve assembly 150 advantageously prevents blood from exiting the sheath 140 before or after a medical instrument has been inserted into the sheath 140. Instead of placing a thumb over the passage 143, or allowing blood to flow from the sheath 140, the physician moves the movable valve assembly 150 over the passage 143, and prevents blood from leaving the sheath 140.

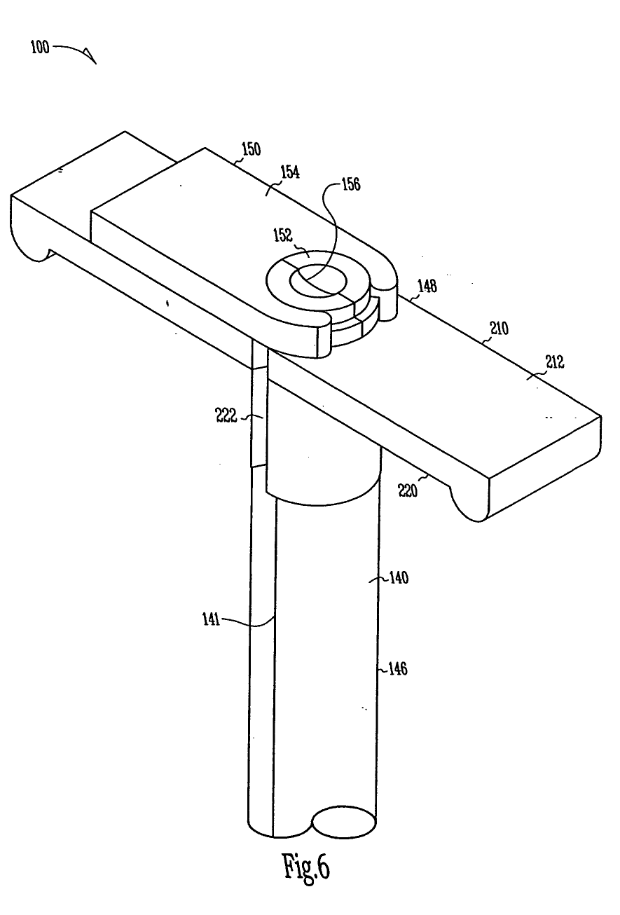

Referring to FIGS. 4 and 7, the sheath 140 optionally further includes locking features such that axial movement between the dilator 120 and sheath 140 is prevented, and optionally further includes anti-rotation features which prevent the dilator 120 from rotating relative to the sheath 140. The dilator 120 includes a rotatable fastener 134 (shown in a cut-away view) rotatably coupled therewith. The rotatable fastener 134 allows for coupling of the dilator 120 to the sheath 140 such that axial movement between the dilator 120 and sheath 140 is prevented. Optionally, the rotatable fastener 134 includes a threaded portion which threadingly engages with the lip 162 of the sheath hub 160.

The dilator 120 optionally includes anti-rotation features, as discussed in U.S. Pat. No. 6,589,262 entitled “Locking Catheter Introducing System” filed on Mar. 31, 2000, and incorporated by reference herein. The anti-rotation features resist and optionally prevent the dilator 120 from rotating relative to the sheath 140. In addition, additional features allow for the anti-rotation features to be overcome, such that the user can selectively rotate the dilator 120 or can selectively lock the rotational movement of the dilator 120. The anti-rotation features, in one option, are disposed on a coupling portion of the dilator 120, and for example include a flat on the coupling portion of the dilator 120.

To assemble the introducing apparatus 100 of FIG. 4, the needle 300 is retractably coupled with the dilator 120. The distal end 122 of the dilator 120 is disposed within the sheath 140 until the dilator hub 126 is proximate to the proximal end 148 of the sheath 140. The rotatable fastener 134 is pressed against the lip 162 of the sheath 140 and the rotatable fastener 134 is rotated. As the fastener 134 is rotated, the dilator 120 becomes further inserted into the sheath 140, and becomes axially fixed to the sheath 140 as the threads engage the lip 162 of the sheath 140. In addition, as the fastener 134 is rotated, the anti-rotation features of the dilator 120 and/or the sheath 140 become seated such that further rotation of the rotatable fastener 134 does not cause rotation of the dilator 120 relative to the sheath 140, even when the fastener 134 is rotated to remove the axial fixation of the dilator 120 relative to the sheath 140.

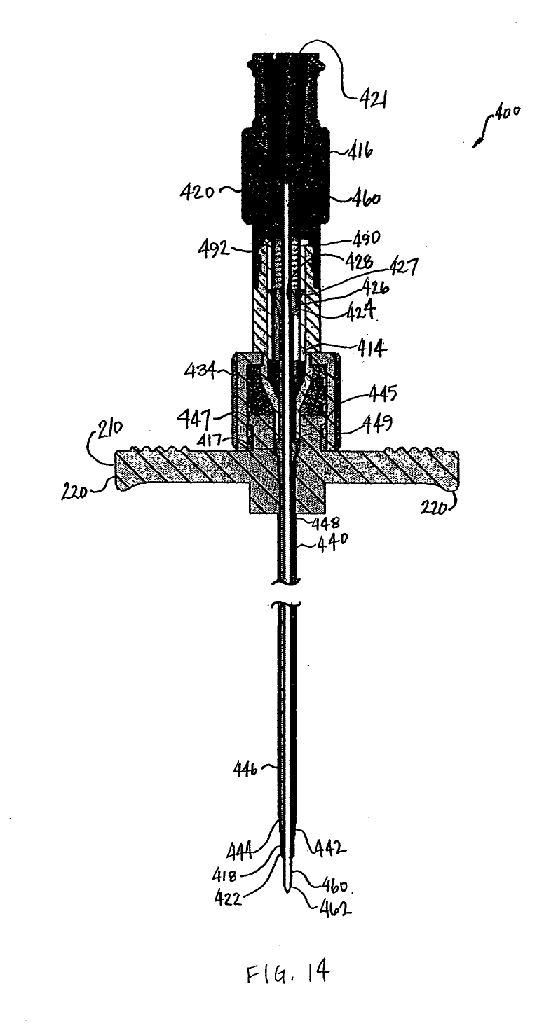

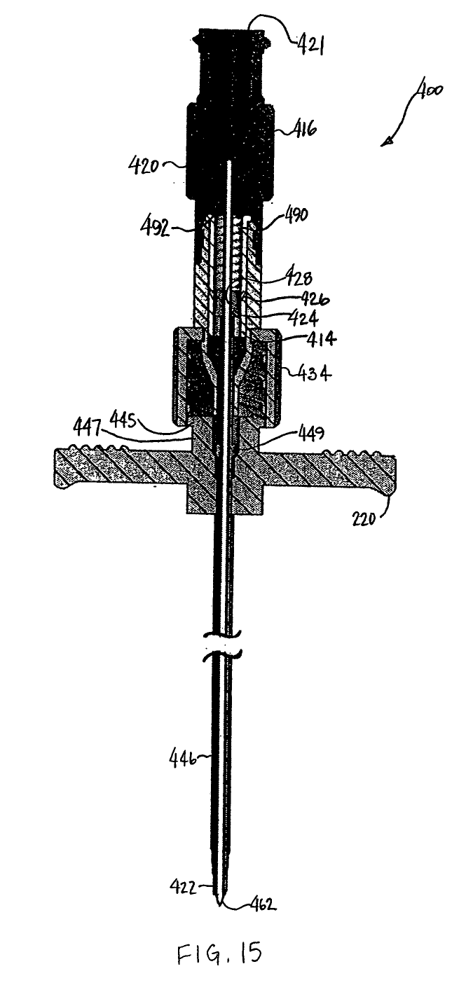

FIGS. 14-20 illustrate additional options for the introducer assembly, and variations that can be combined with the various above-discussed embodiments. Referring to FIGS. 14 and 15, the outer sheath 440, extends from a sheath distal end 442 to a proximal end 448, where the distal end 442 is first inserted into the patient and the proximal end 448 remains outside of the patient. Near the distal end 442 is a tapered portion 444 which provides a transition to a cylindrical portion 446. The outer sheath 440 also includes a passage therethrough which allows for the introduction of the dilator assembly 420 therein. After the introducer assembly 400 has been inserted into a patient, and the dilator assembly 420 is removed, other medical devices, instruments and/or fluids can be easily inserted into and through the outer sheath 440, and introduced into the patient.

At the sheath proximal end 448, the outer sheath 440 includes at least one tab 210 which extends radially outward from the outer sheath 440. In one embodiment, the outer sheath 440 includes two tabs 220, i.e. a first tab and a second tab, which are disposed 480 degrees from each other. In another option, disposed at the sheath proximal end 448 is a sheath shoulder 449. In one option, the sheath shoulder 449 is formed as part of the passage within the outer sheath 440. In another option, the sheath proximal end 448 includes a sheath hub 447, and further optionally includes at least one outer thread 445 on the sheath hub 447.

The outer sheath 440 includes various types of sheaths, for instance, the outer sheath 440 can comprise a sheath which has a strengthening braid of material. Alternatively, the outer sheath 440 includes those which are modified to prevent bends in the outer sheath. In one option, the outer sheath is splittable or otherwise removable from around an instrument disposed therein, without damage to the instrument, for example, through use of the tabs 220. The outer sheath 440 is optionally separable or splittable which prevents disruption to or removal of instruments or devices which have been inserted through the outer sheath 440. Suitable structure to allow the outer sheath to be separable includes score lines, an external a slitting device, a rip cord or strengthening strip running along the longitudinal length of the outer sheath, a weakening which allows the introducer to be ripped apart, or other techniques. It should be noted that the above-discussed features for the outer sheath 440 are optional, and/or interchangeable.

The dilator assembly 420 includes a dilator hub 416 and a dilator sheath 418. The dilator hub 416 having a passage 421 therethrough, and a needle 460 is disposed within the passage 421. The needle 460 is mechanically coupled to the dilator hub 416, and optionally includes any of the above-discussed needles, including, but not limited to, needles with flexible portions. The needle 460 extends to a needle distal end 462, for example a sharpened needle distal end, which is used to pierce the outer skin on a patient, to access, for example, a vein. The needle 460 is disposed within, and extends through the dilator sheath 418.

The dilator sheath 418 is disposed within the passage of the outer sheath 440 where at least a portion of the dilator sheath 418, in one option, is at least temporarily engaged with the outer sheath 440 in an interference fit, or by friction. At least a portion of the dilator sheath 418 is movably disposed within a portion of the dilator hub 416.

The dilator sheath 418 extends from a dilator sheath distal end 422 to a dilator sheath proximal end 424, where the dilator sheath distal end 422 is insertable into a patient. The dilator sheath distal end 422 optionally ends in a tapered end. At the dilator sheath proximal end 424 is a catch 426 having a passage 428 therethrough, where the passage 428 extends through the catch 426 and the dilator sheath 418. The catch 426 moves within the dilator hub 416 from a first position (FIG. 14), through an intermediate position (FIG. 15), and to a final second position (FIG. 16), as further described below. The catch 426 is coupled with the dilator sheath 418, and so the dilator sheath 418 also moves from a first position, through an intermediate position, and to the second position. The catch 426 further includes at least one arm 427, where the arm 427 assists in preventing re-exposure of the needle 460 once it has been covered by the dilator sheath 418. The arm 427 resiliently extends from the catch 426.

The dilator sheath 418 further includes, in one option, a ring 417 projecting out from the dilator sheath 418. The ring 417 is disposed on the dilator sheath 418 between the catch 426 and the dilator sheath distal end 422. When the introducer assembly 400 is assembled and the dilator assembly 420 is coupled with the outer sheath 440, the ring 417 is disposed against the sheath shoulder 449 of the outer sheath 440, and the catch 426 of the dilator sheath 418. The ring 417 is positioned along the dilator sheath 418 such that when the ring 417 is disposed against sheath shoulder 449, and the dilator assembly 420 is coupled with the outer sheath 440, the needle 460 is exposed, and the dilator sheath 418 does not cover the needle 460, as shown in FIG. 14. It should be noted that instead of a ring 417, a projection or recess can be used, and/or the projection can be formed on the outer sheath 440. In one option, the ring 417 is frictionally engaged by the outer sheath 440.

Disposed within the dilator hub 416 is an optional resilient member 490, such as a coil spring. The coil spring is disposed between the dilator hub 416 and a portion of the dilator sheath 418, for example, the coil spring is disposed between a first shoulder 492 within the dilator hub 416 and the catch 426 of the dilator sheath 418. The resilient member 490 is compressed between the dilator hub 416 and at least a portion of the dilator sheath 418 when the dilator sheath 418 is disposed in the first position. In one option, the resilient member 490 is less compressed when the dilator sheath 418 is in the second position than in the first position.

The ring 417 of the dilator sheath 418 and the sheath shoulder 449 assist in forcing the movable dilator sheath 418 against the resilient member 490. The resilient member 490, in one option, assists in preventing re-exposure of the needle 460 once it has been covered by the dilator sheath 418. The dilator hub 416 further includes a second shoulder 414, which optionally mates with the catch 426, for instance, the at least one arm 427. The second shoulder 414, in yet another option, assists in preventing re-exposure of the needle 460 once it has been covered by the dilator sheath 418.

The dilator assembly 420 further includes a fastener which fastens the dilator assembly 420 to the outer sheath 440. In one option, the dilator assembly 420 includes rotatable fastener 434 rotatably coupled therewith. The rotatable fastener 434 allows for coupling of the dilator assembly 420 to the outer sheath 440 such that axial movement between the dilator assembly 420 and outer sheath 440 is prevented. Optionally, the rotatable fastener 434 includes an internally threaded portion which threadingly engages with the outer thread 445 of the outer sheath hub 447.

During use of the introducer assembly 400, the dilator assembly 420 is assembled with the outer sheath 440, and the fastener couples as the dilator assembly 420 with the outer sheath 440 such that axial movement between the dilator assembly 420 and the outer sheath 440 is prevented. When the dilator assembly 420 is coupled with the outer sheath 440, the needle distal end 462 is exposed, and the introducer assembly 400 is configured to be inserted into a patient. In this configuration, the introducer assembly 400 is in the first position, as shown in FIG. 14.

In this position, the ring 417 is disposed against the sheath shoulder 449, and the dilator sheath 418 does not cover the needle distal end 462. Furthermore, the catch 426 of the dilator sheath 418 compresses the resilient member 490 against the first shoulder 492 of the dilator hub 416. In the position shown in FIG. 14, the dilator sheath 418, in one option, is frictionally engaged by the outer sheath 440. Again, the introducer assembly 400 is ready to be inserted into a patient, either over a guidewire, or directly into the vein.

Once the introducer assembly 400 has been properly positioned in the patient, the dilator assembly 420 is removed so that only the outer sheath 440 is disposed within the patient. Additional instruments and/or fluids such as medication can be disposed through the outer sheath 440.

The process of removing the dilator assembly 420 from the outer sheath 440 is important, as the needle 460 is covered, and optionally prevented from further re-use during this process. In another option, as will be discussed further below, a resetting assembly is provided which allows for the dilator sheath 418 to be placed in the first position, after it has been placed in the second position.

As the dilator assembly 420 is removed from the outer sheath 440, the outer sheath 440 retains a portion of the dilator sheath 418, and the catch 426 and the dilator sheath 418 move to the intermediate position relative to the dilator hub 416, as shown in FIG. 15. In one option, the outer sheath 440 retains the dilator sheath 418 by an interference or friction fit between the ring 417 and the sheath shoulder 449. In another option, the outer sheath 440 frictionally engages a portion of the dilator sheath 418.

As the outer sheath 440 retains a portion of the dilator sheath 418 as the dilator assembly 420 is removed from the outer sheath 440, the catch 426 is effectively retained by the outer sheath 440, and the resilient member 490 becomes uncompressed by the catch 426 as the dilator assembly 420 is moved axially away from the outer sheath 440. The resilient member 490, in one option, assists in maneuvering the dilator sheath 418 to cover the needle 460, such that the dilator sheath 418 begins to move away from the dilator hub 416 and toward the needle distal end 462.

As the dilator assembly 420 is moved further axially away from the outer sheath 440, and the resilient member 490 becomes uncompressed, the dilator sheath distal end 422 covers the needle distal end 462 in a second position, as shown in FIG. 16. In this second position, the dilator sheath distal end 422 is disposed over the needle distal end 462, and protects the physician from sticks from a contaminated needle 460. The terms “first”, “intermediate”, and “second” are not intended to be limiting terms, and instead are used to indicate relatively different positions.

To move the dilator sheath 418 to the second position, the user overcomes the friction between the outer sheath 440 (FIG. 14) and the dilator sheath 418 to remove the dilator assembly 420 from the outer sheath 440 (FIG. 14). In this configuration, the dilator sheath 418 and the catch 426 have been moved relative to the dilator hub 416, and the catch 426 has been moved axially past the second shoulder 414 of the dilator hub 416. At least one arm 427 expands, and catches the second shoulder 414, such that the dilator sheath 418 cannot be moved back toward the dilator hub 416, and the needle 460 cannot be re-exposed. In another option, the resilient member 490 assists in preventing the dilator sheath 418 from movement back toward the dilator hub 416, and the needle 460 cannot be re-exposed. It should be noted that any combination of these can also be used to prevent re-exposure of the needle 460. In this second position, the needle 460 has been safely covered by the dilator sheath 41 8, and can be safely disposed of.

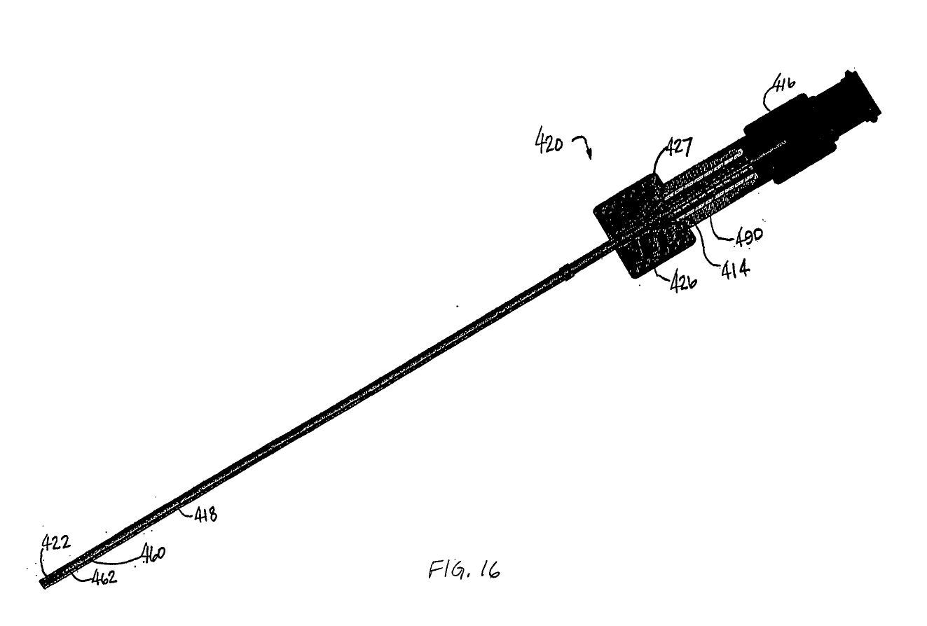

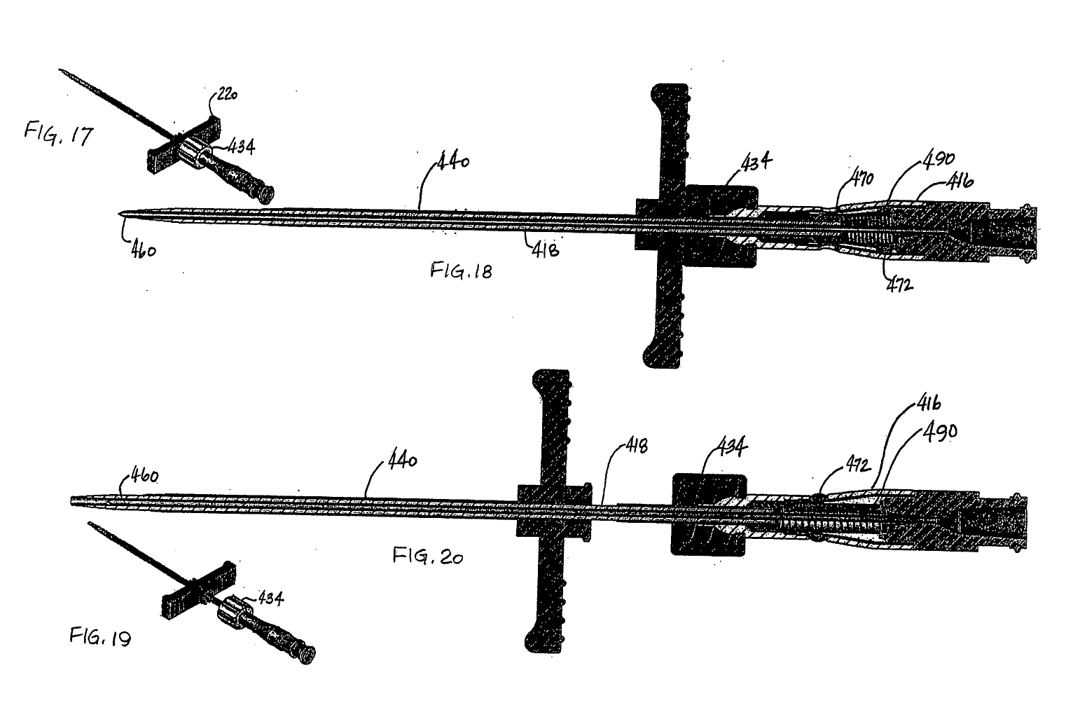

FIGS. 17-20 illustrate another embodiment, and incorporates, but does not require, all of the above-discussed embodiments. Referring to FIGS. 17 and 18, the dilator sheath 418 is disposed in the first position where the needle 460 is exposed, as discussed above. The dilator assembly 420 includes at least one reset assembly. The assembly includes at least one reset member 472 coupled with the dilator sheath 418. The reset assembly allows for the dilator sheath 418 to be positioned back to the first position, after the dilator sheath 418 has been moved to the second position (FIGS. 19 and 20). When the dilator sheath 418 is disposed in the first position and the needle 460 is exposed as shown in FIGS. 17 and 18, the at least one reset member 472 is disposed with the dilator hub 416, or is otherwise contained or covered.

Referring to FIGS. 19 and 20, the dilator sheath 418 is placed in the second position, covering the needle 460, as discussed in the various embodiments above. In this position, the at least one reset member 472 is at least partially exposed through an opening 470 in the dilator hub 416, as shown in FIGS. 19 and 20. To move the dilator sheath 418 back to the first position where the needle 460 is exposed, the physician depresses the at least one reset member 472 to a position within the dilator hub 416, and the physician moves the dilator sheath 418 toward the dilator hub 416, and re-couples the dilator assembly 420 with the outer sheath 440 to expose the needle 460.

During the implant process of the introducer assembly, the physician will stick the vessel with the needle and advance the needle and dilator into the vessel until the dilator distal end is about to enter the opening made by the needle. When the needle has entered the vessel, the pressure of the venous system will cause blood to flow up through the needle into the flash back chamber portion of the dilator hub, which allows the physician one way to visually confirm that the needle has entered the vessel. After verifying the vessel has been accessed by the needle, the dilator is advanced into the vessel. Since the needle is flexible, no guidewire is necessary as the dilator is directed through the vessel. Before, during, or after the dilator advancement through the vessel, the needle is retracted into the dilator. In one option, once the needle has been retracted, it can not be re-extended from the dilator by the user. In one option, the needle is retracted within the dilator using the actuator. In another option, the needle is retracted within the dilator through use of friction, as further discussed below.

The physician optionally further advances the introducer assembly into the vessel. The dilator and retracted needle are removed from the sheath, leaving the sheath in the vessel. A medical device is implanted through the sheath and into the vessel of the patient. The sheath is removed from the medical device without damage to the vessel or the medical device by, for example, peeling or slitting the sheath with a tool.

Use of the apparatus, as described above and including the many variations, includes retractably coupling a needle with a dilator, the dilator extending to a dilator distal end, where the needle extends to a needle distal end and the needle distal end is more flexible than the dilator, and the needle distal end extends beyond the dilator distal end. The method further includes disposing the needle and dilator within a sheath to form an introducing apparatus, and inserting the introducing apparatus into a body.

Several options for the method are as follows. For example, in one option, the method further includes retracting the needle within the dilator, and removing the needle and the dilator from the sheath. In another option, the method further includes preventing re-extension of the needle from the dilator. In yet another option, the method further includes removing the dilator and needle from the sheath, inserting an instrument through the sheath, and separating the sheath from the instrument without damage to the instrument. A valve is coupled with the sheath in another option.

The present introducing assembly requires fewer parts, includes fewer steps than the traditional Seldinger Technique, and is less expensive to manufacture, and insert into a patient. A further benefit is that once the needle is retracted, the dilator cannot accidentally stick the implanter. In addition, the mechanism which prevents re-extension prevents the introducer used on one patient from being used on another patient. Since the guidewire is no longer necessary, fewer steps are needed to introduce an instrument into a patient, resulting in a faster process, and less trauma to a patient. Yet another advantage is that a more effective seal is made around the catheter or medical instrument since the device which retains or supports the valve flexes, for example, as instruments are inserted therethrough. The introducing assembly can be manufactured in a wide variety of sizes, and allows for any type of medical device or fluid to be disposed therethrough.

It is to be understood that the above description is intended to be illustrative, and not restrictive. Many other embodiments will be apparent to those of skill in the art upon reading and understanding the above description. It should be noted that embodiments or portions thereof discussed in different portions of the description or referred to in different drawings can be combined to form additional embodiments of the present invention. The scope of the invention should, therefore, be determined with reference to the appended claims, along with the full scope of equivalents to which such claims are entitled.

What is claimed is:

1. An introducing apparatus comprising:
a tubular sheath extending from a sheath proximal end to a sheath distal end, the sheath sized to receive a dilator therethrough;
the dilator extending from a dilator proximal end to a dilator distal end;
a needle disposed within the dilator, the needle extending from a needle proximal end to a needle distal end and including an intermediate portion therebetween, at least a portion of the needle is at least as flexible as the dilator;
the needle distal end extending out of the dilator distal end in a first position, the needle distal end retracted within the dilator distal end in a second position; and
means for retracting the needle distal end within the dilator.

2. The introducing apparatus as recited in claim 1, wherein the sheath is separable without damage to an instrument inserted therethrough.

3. The introducing apparatus as recited in claim 1, wherein the needle distal end is more flexible than the dilator.

4. The introducing apparatus as recited in claim 1, wherein the needle distal end has the same or more flexibility as the dilator.

5. The introducing apparatus as recited in claim 1, wherein the intermediate portion of the needle comprises a flexible coil.

6. The introducing apparatus as recited in claim 1, wherein the intermediate portion and the needle distal end are flexible, and the intermediate portion and the needle distal end are formed of a unitary structure of nitinol.

7. The introducing apparatus as recited in claim 1, wherein the needle proximal end includes a member sized and shaped to prevent re-extension of the needle.

8. The introducing apparatus as recited in claim 1, further comprising a locking mechanism configured to temporarily lock the sheath with the dilator.

9. The introducing apparatus as recited in claim 1, wherein the dilator further includes a blood flashback chamber.

10. The introducing apparatus as recited in claim 1, further including a valve coupled with the sheath.

11. The introducing apparatus as recited in claim 10, wherein the valve comprises a sliding valve assembly slidingly engaged with the at least one tab, the sliding valve adapted to slide from a first position to a second position, in the first position the sliding valve disposed through the longitudinal axis of the sheath, in the second position the sliding valve disposed away from the longitudinal axis of the sheath.

12. An introducing apparatus comprising:
an elongate tubular sheath extending from a sheath proximal end to a sheath distal end, the sheath sized to receive a dilator therethrough;
the dilator extending from a dilator proximal end to a dilator distal end;
a needle disposed within the dilator, the needle extending from a needle proximal end to a needle distal end and including an intermediate portion therebetween, at least a portion of the needle is flexible;
the needle distal end extending out of the dilator distal end in a first position, the needle distal end retracted within the dilator distal end in a second position; and
a needle retraction mechanism associated with the needle and the dilator, and the needle is retractably coupled with the dilator.

13. The introducing apparatus as recited in claim 12, wherein the needle includes a catch sized and shaped to prevent extension of the distal end of the needle from the dilator distal end.

14. The introducing apparatus as recited in claim 12, wherein the needle distal end is more flexible than the dilator.

15. The introducing apparatus as recited in claim 12, wherein the dilator further includes a blood flashback chamber and a gas permeable filter.

16. The introducing apparatus as recited in claim 12, further including a valve coupled with the sheath.

17. The introducing apparatus as recited in claim 16, wherein the valve comprises a sliding valve assembly slidingly engaged with the at least one tab, the sliding valve adapted to slide from a first position to a second position, in the first position the sliding valve disposed through the longitudinal axis of the sheath, in the second position the sliding valve disposed away from the longitudinal axis of the sheath.

18. A method comprising:
disposing a needle within a dilator;
retractably coupling a needle with a dilator, the dilator extending to a dilator distal end, where the needle extends to a needle distal end and at least a portion of the needle is more flexible than the dilator, and the needle distal end extends beyond the dilator distal end in a first position; and
disposing the needle and dilator within a sheath to form an introducing apparatus.

19. The method as recited in claim 18, further comprising retracting the needle distal end within the dilator.

20. The method as recited in claim 18, further comprising retaining the dilator with the sheath and retracting the needle distal end within the dilator.

21. The method as recited in claim 19, further comprising preventing re-extension of the needle from the dilator.

22. The method as recited in claim 18, further comprising removing the dilator and needle from the sheath, inserting an instrument through the sheath, and separating the sheath from the instrument without damage to the instrument.

23. The method as recited in claim 18, further comprising coupling a valve with the sheath.

24. The method as recited in claim 18, further comprising coupling a coil between a position adjacent to the needle distal end and a needle proximal end to form a flexible portion therebetween.
